(19) United States
(12) Patent Application Publication
(10) Pub. No.: US 2026/0207377 A1
(43) Pub. Date: Jul. 23, 2026

(54) Modular Ophthalmic Delivery System With Replaceable Pod and Flow Control Mechanism

(71) Applicant: Robert J Behan, Fort Wright, KY (US)

(72) Inventor: Robert J Behan, Fort Wright, KY (US)

(73) Assignee: Bullseye Dropper, LLC, Crescent Springs, KY (US)

(21) Appl. No.: 19/230,963

(22) Filed: Jun. 6, 2025

Related U.S. Application Data
(60) Provisional application No. 63/664,843, filed on Jun. 27, 2024.

(51) Int. Cl. A61F 9/00

(52) U.S. Cl. CPC A61F 9/0026

(57) ABSTRACT

An ophthalmic delivery system includes a reusable body defining an internal cavity configured to receive a replaceable consumable containing ophthalmic liquid. The body includes first and second feet configured to stabilize the body against a user's face adjacent an eye, and an actuation mechanism configured to transmit force to the consumable to dispense ophthalmic liquid through a fluid outlet of the consumable. The internal cavity may include predetermined interior geometry configured to receive the consumable in a fixed operational position, align the consumable with the actuation mechanism, and inhibit rotation of the consumable. The body may include an entry port, an exit port, and a retention shelf configured to limit insertion depth of the consumable. When seated in the body, the consumable is positioned to dispense ophthalmic liquid along a protected, standoff delivery path.

### INCORPORATION BY REFERENCE

This application claims the benefit of U.S. Provisional Patent Application No. 63/664,843, filed Jun. 27, 2024, and incorporates by reference, in their entireties, the disclosures of said provisional application, U.S. Pat. No. 10,299,959 B2, and U.S. Design Patent No. D765,834 S.

### FIELD OF THE INVENTION

The present invention relates generally to ophthalmic drug-delivery devices and methods. More specifically, it relates to a modular ophthalmic delivery system comprising a reusable body configured to receive a replaceable consumable containing an ophthalmic liquid, and an actuation mechanism carried by the body for dispensing the ophthalmic liquid.

### BACKGROUND OF THE INVENTION

Conventional eyedropper bottles, whether over-the-counter or prescription, commonly require a user to position a bottle tip above the eye while tilting the head backward and manipulating the eyelids. This process can be unstable, inaccurate, and intimidating, particularly because the user must aim a falling drop toward the ocular surface.

Conventional administration may be especially difficult for individuals with limited dexterity, reduced vision, tremors, arthritis, weakness, or fear of objects approaching the eye. Poor visibility, hand movement, blinking, or flinching may result in missed doses, misdirected drops, repeated attempts, or uncertainty whether medication actually reached the eye. These challenges may cause some patients to delay or forego treatment, reducing adherence and potentially worsening the underlying condition.

Conventional eyedroppers may also dispense drop volumes larger than the volume the eye is able to retain. For example, conventional devices may dispense drops in a range of about 30 μL to 60 μL, whereas the eye may retain about 10 μL. Excess liquid may drain down the face or into the tear duct, resulting in waste and possible systemic absorption.

Because conventional eyedropper bottles are generally used independently of a stabilizing body or controlled standoff structure, the bottle tip may be positioned inconsistently relative to the eye during use. Inadvertent contact between the bottle tip and the ocular surface, eyelashes, eyelids, or surrounding skin may cause discomfort or injury and may transfer contaminants to the bottle tip, potentially affecting subsequent administrations.

Conventional ophthalmic delivery systems also generally do not provide a controlled interface between a delivery device and a replaceable liquid container. As a result, containers of varying geometry or configuration may be used without defined alignment, retention, or actuation characteristics. Such variability may affect positioning of the fluid outlet relative to the eye, consistency of dose delivery, contamination control, and user safety.

In addition, conventional single-use and multi-use bottles may require larger liquid volumes to provide a given number of administrations because of their relatively larger drop size. A container dispensing 40 μL drops, for example, may require approximately four times the liquid volume of a container dispensing 10 μL drops to provide the same number of doses. Larger liquid volumes may in turn require larger containers, increased material usage, and waste.

### SUMMARY OF THE INVENTION

In one aspect, the invention provides an ophthalmic delivery system comprising a reusable body configured to stabilize against a user's face adjacent an eye, a replaceable consumable containing an ophthalmic liquid and including a fluid outlet, and an actuation mechanism carried by the body, wherein the actuation mechanism is configured to cause dispensing of ophthalmic liquid through the fluid outlet of the consumable upon actuation while maintaining a protected, standoff delivery path, and wherein the consumable is insertable into the body by a user prior to use and removable from the body after the contents of the consumable are depleted. In one embodiment, the consumable comprises a pre-filled pod containing the ophthalmic liquid, a flow-control hub coupled to the pod and including the fluid outlet, and a protective cap.

### BRIEF DESCRIPTION OF THE DRAWINGS

The accompanying drawings, which are incorporated in and constitute a part of this specification, illustrate exemplary embodiments of the invention and, together with the detailed description, serve to explain the structure and operation of the disclosed system.

FIG. 1 illustrates the ophthalmic delivery system 100 in use, positioned on a user's face. The system is shown stabilized against the brow and cheek while a drop of ophthalmic liquid 12 is dispensed from the fluid outlet of the consumable toward the user's eye. Actuation mechanism 2 is shown being actuated by the user's forefinger 13. The user's hand 14, nose 15, and eye 16 are shown in broken lines.

FIG. 2 shows ophthalmic delivery system 100 being held by the user's hand 14 during actuation. A drop of ophthalmic liquid 12 is shown being dispensed from the fluid outlet of the consumable as actuation mechanism 2 is pressed. Body 1, flow-control hub 5, first foot 7, and second foot 8 are visible. The user's forefinger 13 is shown in broken lines.

FIG. 3 is a perspective view of ophthalmic delivery system 100, showing reusable body 1, actuation mechanism 2, flow-control hub 5, and protective cap 4 aligned for attachment. First foot 7 and second foot 8 are visible.

FIG. 4 is an exploded perspective view of ophthalmic delivery system 100, illustrating actuation mechanism 2 in an open position and pod 3, flow-control hub 5, and protective cap 4 aligned for insertion through entry port 6 of reusable body 1. First foot 7 and second foot 8 are visible.

FIG. 5 is an exploded perspective view showing pod 3 and flow-control hub 5 aligned for assembly and insertion into body 1. First foot 7 and second foot 8 are visible.

FIG. 6 is a side view of fully assembled ophthalmic delivery system 100, shown with protective cap 4 secured and actuation mechanism 2 in a closed position. Body 1, first foot 7, and second foot 8 are visible.

FIG. 7 is a cutaway side view of ophthalmic delivery system 100, showing pod 3 and flow-control hub 5 seated within body 1 and secured in place with actuation mechanism 2 in a closed position. Entry port 6, first foot 7, and second foot 8 are visible.

FIG. 8 is a cutaway side view similar to FIG. 7, except that actuation mechanism 2 is shown in an open position to permit insertion or removal of pod 3 and flow-control hub 5 through entry port 6. Body 1, first foot 7, and second foot 8 are visible.

FIG. 9 is an exploded side view of representative consumable 200. In one embodiment, consumable 200 comprises a container designed to hold liquid known throughout as pod 3 and flow-control hub 5 shown in exploded relation, with connection neck 10 of pod 3 and inlet 11 of flow-control hub 5 visible. In one embodiment, flow-control hub 5 includes embedded cannula 9. In another embodiment, flow-control hub 5 includes an integral protruding portion serving the same purpose as cannula 9. Embodiments of the consumable are described in a companion application by the same inventor.

FIG. 10 is an exploded perspective view of side-actuation embodiment 300 of ophthalmic delivery system 300, showing reusable body 1 with entry port 6, first foot 7, second foot 8, actuation mechanism 21 positioned on a lateral side of body 1, exit port 18, retention shelf 19, and entry port cap 20 shown separated from entry port 6.

FIG. 11 is a perspective view of the side-actuation embodiment of ophthalmic delivery system 300, showing reusable body 1, entry port cap 20 positioned at entry port 6, actuation mechanism 21, flow-control hub 5 protruding through exit port 18, retention shelf 19, first foot 7, and second foot 8.

FIG. 12 illustrates side-actuation embodiment 300 in use, shown stabilized against a user's face with first foot 7 and second foot 8 contacting facial landmarks on opposite sides of the eye socket. Actuation mechanism 21 is shown being operated by the user's hand 14, with the user's forefinger 13 and thumb 22 engaging opposite portions of body 1. Expelled liquid 12 is shown in transit toward eye 16. Flow-control hub 5, retention shelf 19, and entry port cap 20 are visible. Human anatomical references are shown in broken lines.

FIG. 13 is a perspective view of T-hinge embodiment 400, showing body 1, entry port 6, T-shaped living hinge actuation mechanism 23, exit port 18, retention shelf 19, first foot 7, second foot 8, and entry port cap 20 shown separated from entry port 6.

FIG. 14 is a side view of representative consumable 200, showing pod 3, flow-control hub 5, and protective cap 17 in an assembled configuration. Exterior geometry feature 3a is shown on pod 3, exterior geometry feature 3b is shown at a lower transition region of pod 3, exterior geometry feature 5a is shown as a collar portion of flow-control hub 5, and exterior geometry feature 17a is shown on protective cap 17. In one embodiment, exterior geometry features 3a, 3b, 5a, and 17a comprise alignment, keying, and retention surfaces configured to complement predetermined interior geometry of a body cavity and thereby position consumable 200 in a fixed operational position within the body.

FIG. 15 is a cutaway view of representative consumable 200 seated within body 1, showing consumable 200 received in an internal cavity of body 1 in a fixed operational position. Pod 3, flow-control hub 5, entry port 6, protective cap 17, exit port 18, and retention shelf 19 are visible. Retention shelf 19 is shown engaging consumable 200 at a predetermined depth.

FIG. 16 is a composite view showing representative consumable 200 and a cutaway view of T-hinge embodiment 400. Consumable 200 is shown with exterior geometry feature 3a on pod 3, exterior geometry feature 3b at a lower transition region of pod 3, exterior geometry feature 5a on flow-control hub 5, and exterior geometry feature 17a on protective cap 17. The cutaway view of embodiment 400 shows interior geometry features of body 1 including narrowed wall region 24, inward protrusion 25 and limiting feature 28, both associated with T-shaped living hinge actuation mechanism 23, body cavity 27, retention shelf 19, interior geometry 26 of retention shelf 19, and exit port 18. In one embodiment, narrowed wall region 24 is configured to receive pod 3 in a predetermined orientation defined by exterior geometry feature 3a. Exterior geometry feature 3b provides a transition surface facilitating insertion of pod 3 past narrowed wall region 24. In one embodiment, inward protrusion 25 is positioned to engage pod 3 and transmit inward movement of T-shaped living hinge actuation mechanism 23 to pod 3 during actuation. Interior geometry 26 of retention shelf 19 is configured to engage exterior geometry feature 5a of flow-control hub 5 to retain consumable 200 within body cavity 27 and inhibit rotation of consumable 200 within body 1. Limiting feature 28 is configured to limit travel of actuation mechanism 23 during dispensing. Exit port 18 has a predetermined geometry configured to admit a correspondingly configured protective cap 17 having exterior geometry 17a.

### DETAILED DESCRIPTION OF THE INVENTION

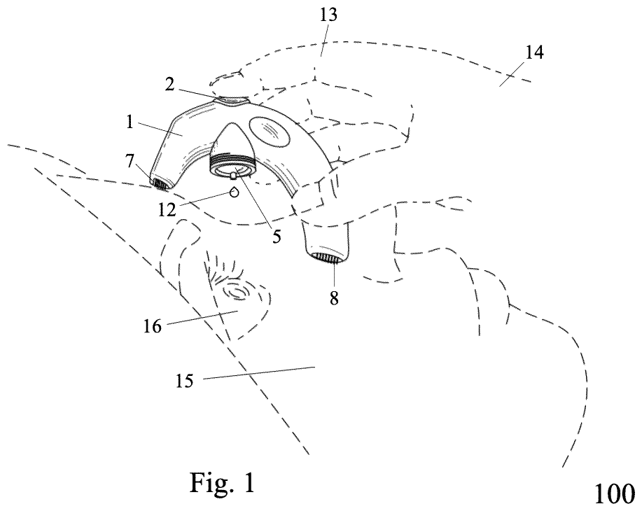

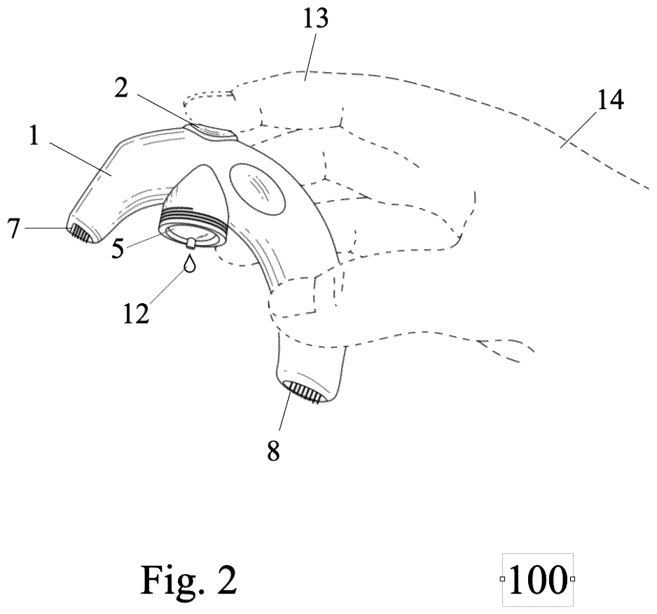

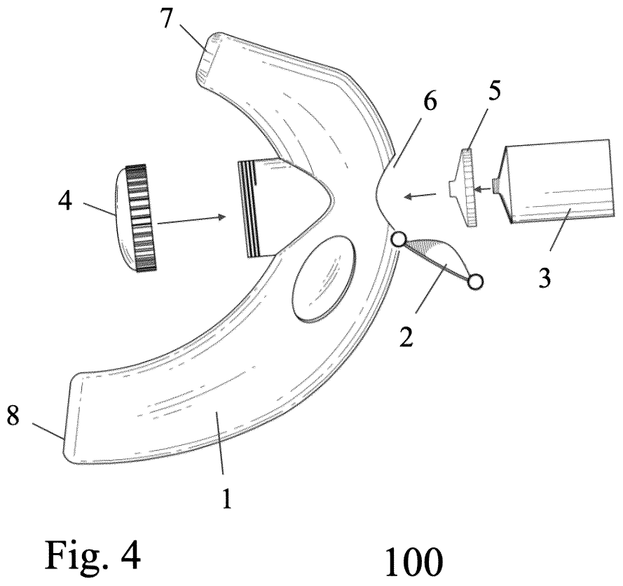

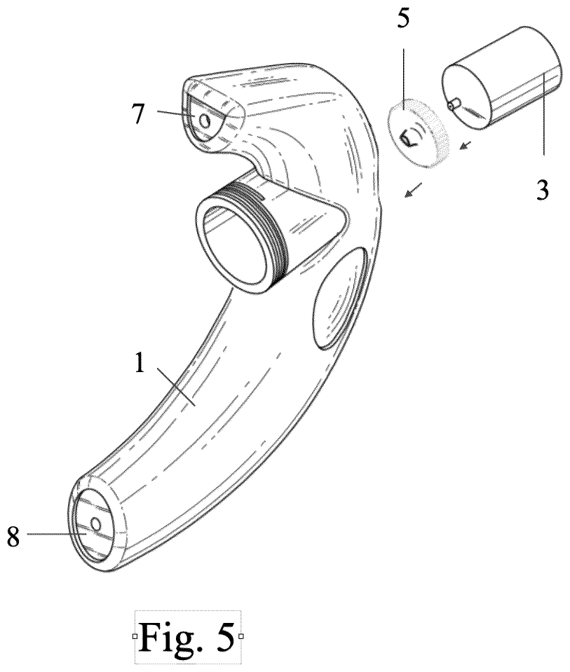

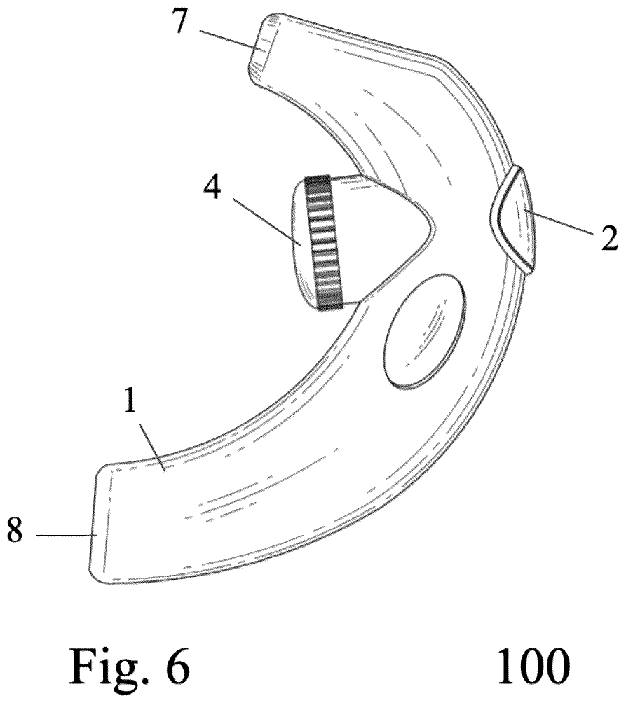

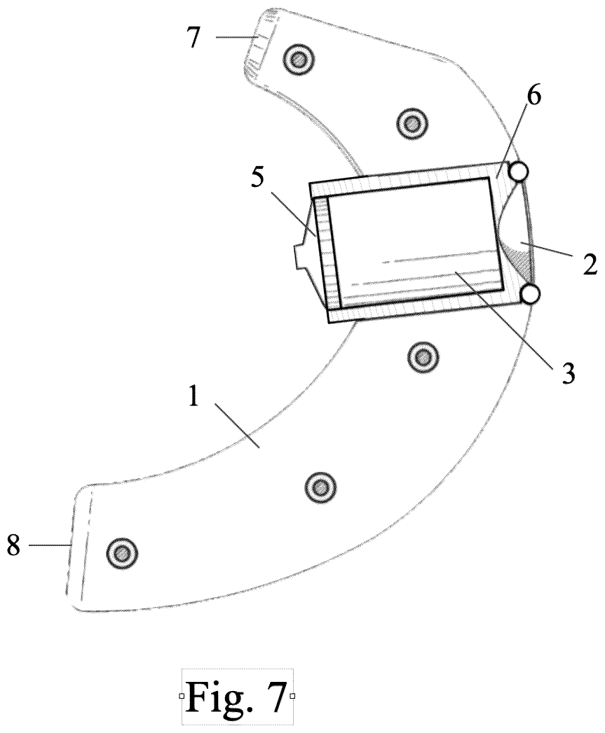

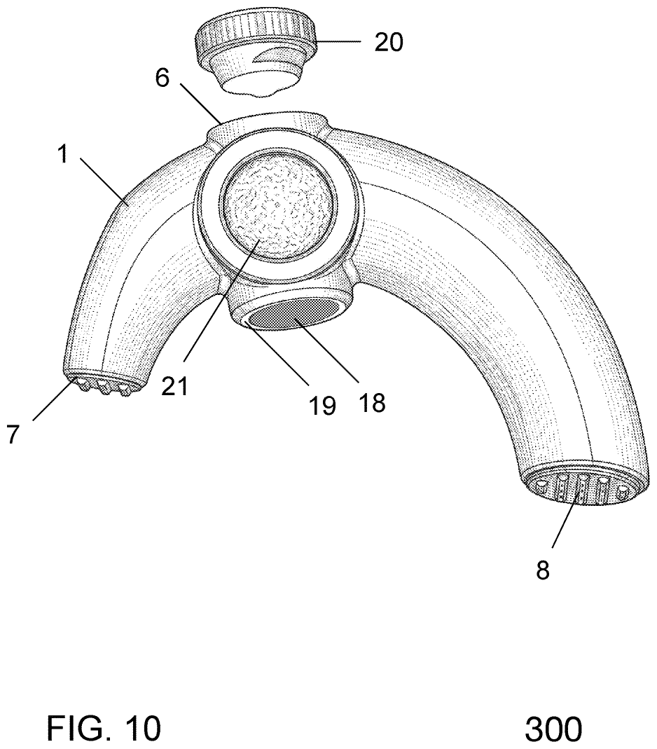

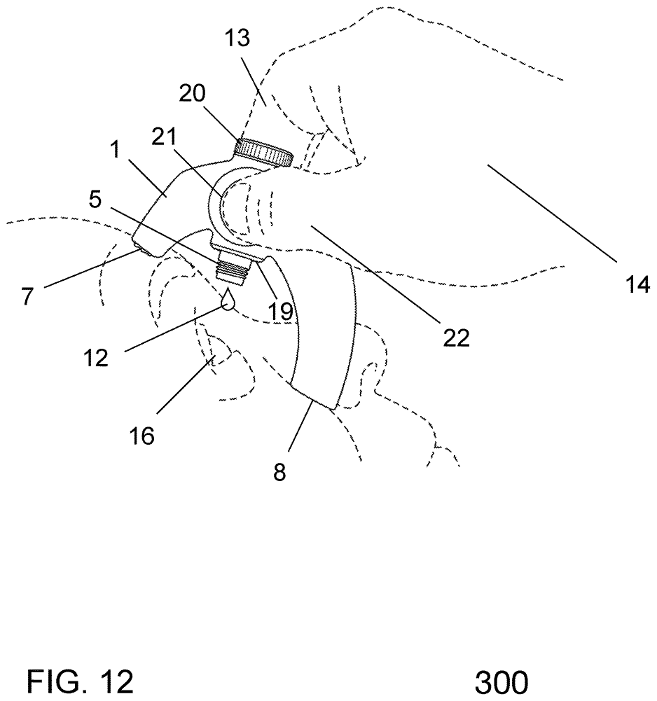

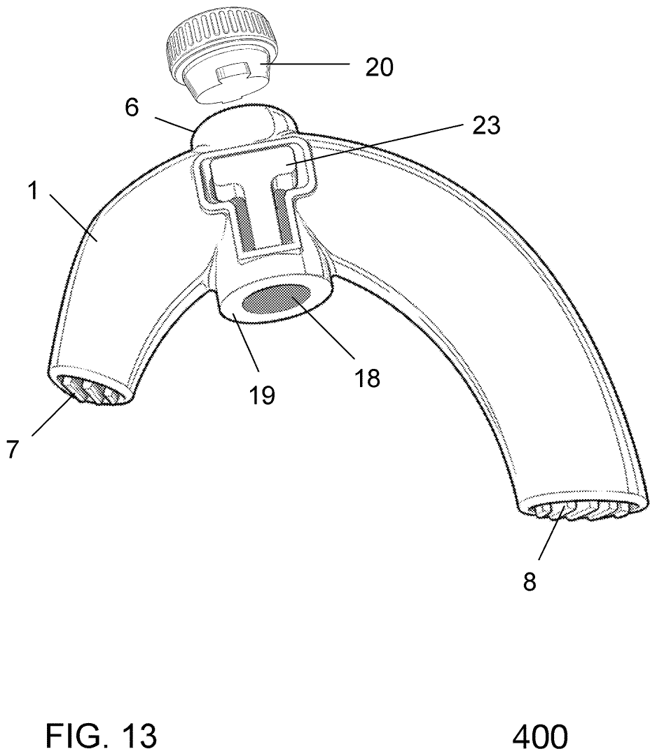

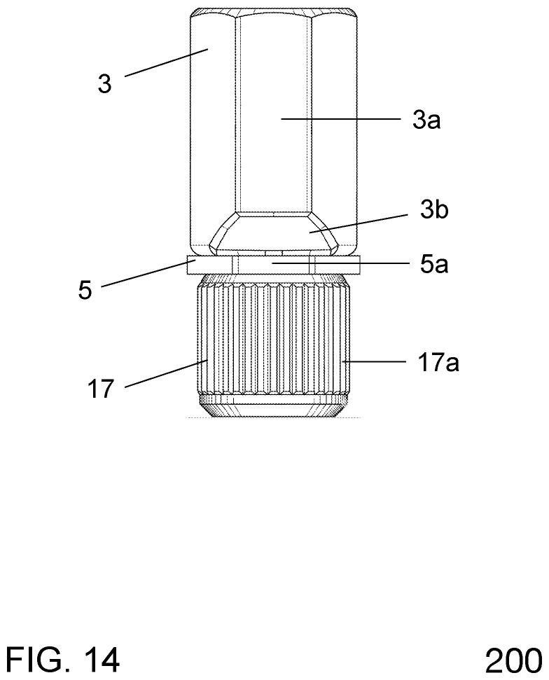

With reference to FIGS. 1-8, ophthalmic delivery system 100 comprises a durable, reusable body 1 configured to receive a replaceable consumable through port 6 of body 1. With reference to FIGS. 10-12, ophthalmic delivery system 300 comprises body 1 having separately identified entry port 6 and exit port 18. With reference to FIGS. 13, 15, and 16, ophthalmic delivery system 400 comprises body 1 having separately identified entry port 6 and exit port 18. In one embodiment, the consumable comprises a resilient deformable pod 3 containing ophthalmic medication and a flow-control hub 5 providing a fluid flow path. In ophthalmic delivery system 100, protective cap 4 is attachable to and detachable from body 1. In the embodiments shown in FIGS. 14-16, protective cap 17 is attachable to and detachable from flow-control hub 5.

As shown in FIGS. 1-8, 10-13, 15, and 16, body 1 may have an arched configuration defining a first leg terminating in first foot 7 and a second leg terminating in second foot 8. First foot 7 and second foot 8 are configured to contact facial regions on opposite sides of the eye socket so as to stabilize body 1 relative to the eye during use. In one embodiment, first foot 7 is configured to contact the face above the eye socket and second foot 8 is configured to contact the face below the eye socket. In one embodiment, the leg terminating in first foot 7 is shorter than the leg terminating in second foot 8, positioning the fluid outlet in closer proximity to the lower portion of the eye socket and reducing the degree of head tilt required during administration. In one embodiment, the legs are asymmetrical. In another embodiment, the legs are substantially symmetrical. The size, contour, spacing, and relative positioning of first foot 7 and second foot 8 may be selected to provide stable engagement with the user's face while maintaining the consumable in a protected, standoff relationship relative to the eye.

The apex of the underside of arched body 1 is dimensioned and positioned such that, when feet 7 and 8 are stabilized against the user's facial landmarks during dosing, the distal tip of the consumable, including the fluid outlet and any surrounding protective structure, remains spaced away from the ocular surface, eyelashes, and surrounding periocular tissue. This spacing reduces the likelihood of inadvertent contact with the eye or surrounding skin and reduces contamination risk. In a preferred embodiment, the distal tip of the consumable does not extend beyond a plane that is tangential to both first foot 7 and second foot 8 when the consumable is fully seated within body 1. The internal cavity of body 1 is dimensioned such that the consumable is positioned within the arch span defined between first foot 7 and second foot 8, maintaining this recessed relationship across properly configured consumables.

In use, a user places first foot 7 and second foot 8 against facial landmarks adjacent the eye, aligns body 1 relative to the eye, and actuates actuation mechanism 2, 21, or 23 to dispense ophthalmic liquid from the consumable toward the eye. In one embodiment, engagement of body 1 with the user's face permits single-handed administration. In one embodiment, second foot 8 is positioned so the user may apply gentle downward tension to the lower eyelid, exposing the lower conjunctival sac for eye drop delivery. Stabilization of body 1 against the face and positioning of the fluid outlet along the protected, standoff delivery path facilitate repeatable administration of ophthalmic liquid. The combined facial stabilization and direct alignment of the fluid outlet of the consumable with the eye allow the user to avert their gaze away from the falling eye drop, thereby reducing the blink reflex during drop administration.

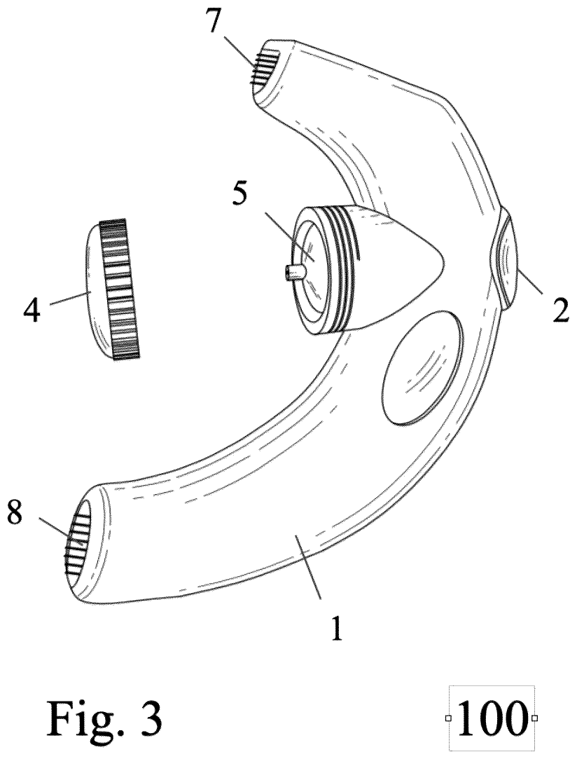

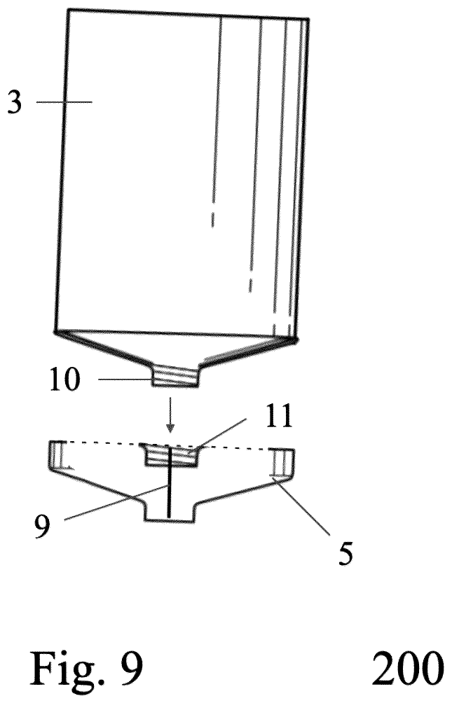

As shown in FIG. 9, in one embodiment, the consumable comprises pod 3 configured to contain an ophthalmic liquid and flow-control hub 5 coupled to pod 3. In one embodiment, pod 3 is formed from a resilient deformable material, such as LDPE, and is formed, filled, and sealed using blow-fill-seal technology such that the ophthalmic liquid is hermetically sealed within pod 3 prior to use. In another embodiment, the consumable may be a unitary structure integrating a liquid reservoir and a fluid outlet. Where separate components are used, pod 3 and flow-control hub 5 may be coupled at connection neck 10 and inlet 11 by threaded engagement, snap-fit engagement, press-fit engagement, luer lock engagement, or other suitable connection. In one embodiment, flow-control hub 5 includes embedded cannula 9 configured to extend through inlet 11 and pierce pod 3 at the base of connection neck 10 so as to place the interior of pod 3 in fluid communication with flow-control hub 5. In another embodiment, flow-control hub 5 includes an integral protruding portion serving the same purpose as cannula 9. Once pod 3 is pierced, depression of an outer wall of pod 3 by an actuator of body 1 causes ophthalmic liquid to flow through flow-control hub 5 and exit through the fluid outlet. After actuation, pod 3 rebounds toward its original shape. In the embodiment shown in FIG. 14, consumable 200 further includes protective cap 17 removably attachable to flow-control hub 5. In the embodiment shown in FIGS. 3, 4, and 6, protective cap 4 is removably attachable to body 1. When pod 3 is depleted of ophthalmic liquid, consumable 200, including pod 3, flow-control hub 5, and, where present, protective cap 17, as shown in FIG. 14, is removed from body 1 by the user through port 6 using the user's fingers or hand and is discarded. A replacement consumable 200 is then inserted into body 1 for subsequent use. Embodiments of the consumable are described in a companion application by the same inventor.

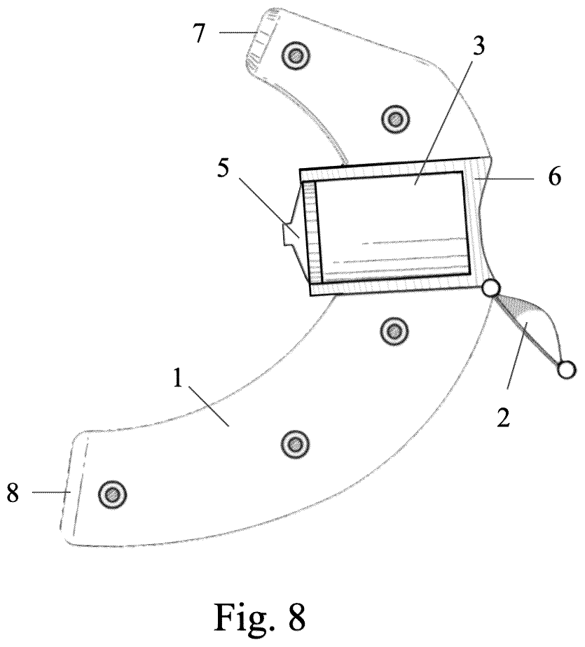

The actuation mechanism carried by body 1 is configured to transmit force to the consumable so as to cause dispensing of ophthalmic liquid through the fluid outlet of the consumable. The actuation mechanism may comprise a mechanical button, a mechanical switch, an electrically actuated actuator, a manually depressible button, a flexible membrane, or other mechanism configured to deform the consumable. In one embodiment, shown in FIGS. 1-8, actuation mechanism 2 is positioned at an upper region of body 1 and is operable in a closed position, as shown in FIGS. 1, 2 and 7, to dispense ophthalmic liquid. In FIG. 8, actuation mechanism 2 is shown in an open position, in which actuation mechanism 2 is pivoted about a hinge to permit insertion or removal of the consumable through port 6.

In a second embodiment, shown in FIGS. 10-12, actuation mechanism 21 is positioned on opposite lateral sides of body 1, permitting actuation by the user's fingers while body 1 remains stabilized against the face. Actuation mechanism 21 is implemented as a resilient structure configured to deform under lateral finger pressure and return toward a resting position upon release. Lateral finger pressure on actuation mechanism 21 causes a protrusion associated therewith to transmit force to the consumable, thereby causing ophthalmic liquid to be dispensed from the fluid outlet of the consumable. In some embodiments of the side-actuation configuration, actuation mechanism 21 is configured to deliver a calibrated dose per actuation. Body 1 in the side-actuation embodiment including resilient structure 21 is designated as 300-series assembly 300.

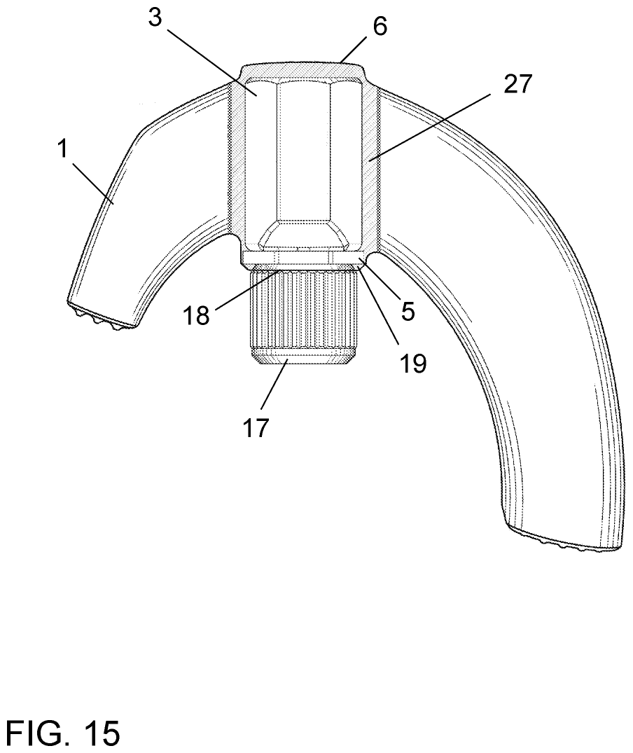

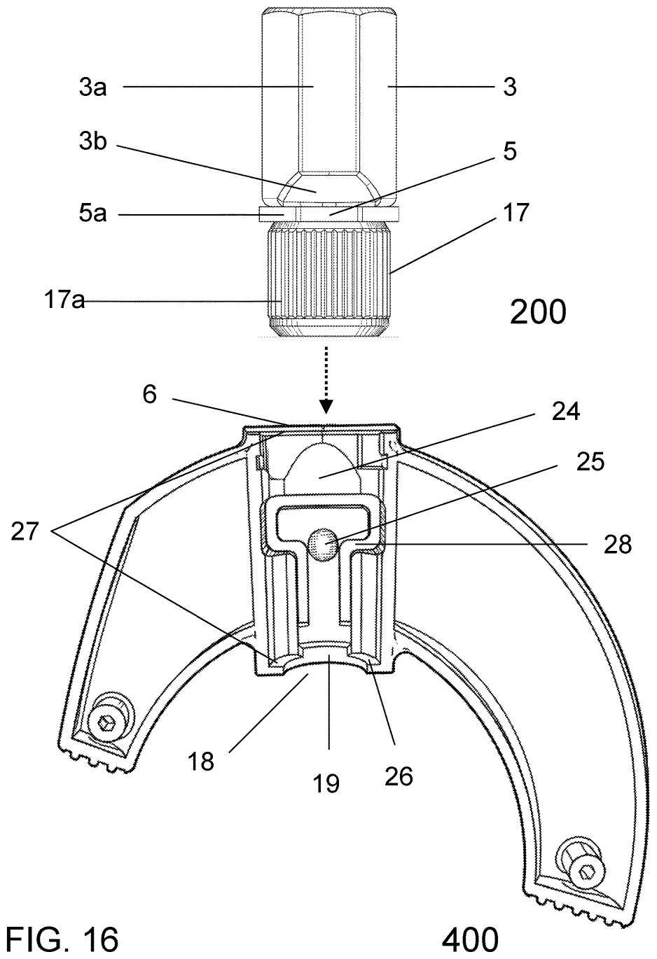

In FIGS. 10, 11, 13, 15, and 16, entry port 6 and exit port 18 are separately defined in embodiments 300 and 400. Retention shelf 19 is configured to limit insertion depth of the consumable. In FIGS. 15 and 16, body cavity 27 is configured to receive and hold the consumable between entry port 6 and exit port 18. In one embodiment, body cavity 27 includes keying elements configured to cooperate with complementary geometry of the consumable so as to position the consumable in a predetermined orientation within body 1 and permit receipt only of a correspondingly configured consumable.

In a further embodiment, shown in FIGS. 13, 15, and 16, T-hinge embodiment 400 includes T-shaped living hinge actuation mechanism 23. Actuation mechanism 23 is configured to deflect inward under manual pressure and to return toward a resting position upon release. In one embodiment, inward movement of actuation mechanism 23 is transmitted by inward protrusion 25, shown in FIG. 16, to deform pod 3 thereby causing dispensing of ophthalmic liquid from the consumable. In this embodiment, body 1 includes entry port 6, exit port 18, and retention shelf 19 for receiving and positioning the consumable within body cavity 27. In one embodiment, limiting feature 28 is configured to limit travel of T-shaped living hinge actuation mechanism 23, thereby further controlling dispensing of ophthalmic liquid.

In one embodiment, FIG. 15 shows the consumable seated within body 1 with flow-control hub 5 engaging retention shelf 19. FIG. 16 shows representative consumable 200 positioned for insertion into body cavity 27 through entry port 6. The consumable is installed into body 1 by removing entry port cap 20 from entry port 6, inserting the protective cap 17 end of the consumable into entry port 6 first, and advancing the consumable through the internal cavity of body 1 by applying force to pod 3 until the consumable comes to rest on retention shelf 19. When fully seated, protective cap 17 protrudes through exit port 18. Entry port cap 20 is then replaced to retain the consumable within body 1 during use. Removal of a depleted consumable is performed by removing entry port cap 20 and advancing protective cap 17 until pod 3 protrudes from entry port 6, whereupon the user grasps pod 3 and withdraws the consumable from body 1. Entry port cap 20 may then be replaced. In another embodiment, as shown in FIGS. 1-9, the consumable is inserted into and removed from body 1 through port 6 by opening actuation mechanism 2 to access the internal cavity, positioning the consumable within body 1, and closing actuation mechanism 2 to secure the consumable in position.

In other embodiments, body 1 may include a locking structure configured to inhibit or prevent actuation of the actuation mechanism when not in use. The locking structure may be manually engageable or disengageable by a user and may include a switch, latch, detent, slide, cover, rotation feature, or other mechanism configured to block, restrict, or otherwise inhibit movement of the actuation mechanism to reduce inadvertent dispensing of ophthalmic liquid.

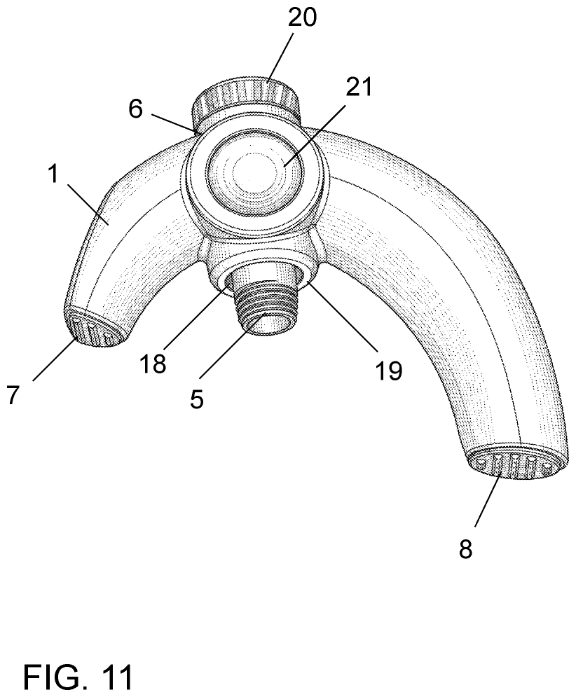

Internal cavity 27 of body 1 has predetermined interior geometry configured to receive and retain the consumable in a fixed operational position through complementary engagement between the interior geometry of body 1 and exterior geometry of the consumable. The fixed operational position is the position at which the consumable is fully seated within the cavity, retained against displacement, and aligned with the actuation mechanism for actuation. As used herein, a “properly configured consumable” means a replaceable unit bearing exterior geometry corresponding to the predetermined interior geometry of the internal cavity of body 1, including the cavity wall profile. When a properly configured consumable is received within body 1, the complementary engagement provides full seating, retention, alignment with the actuation mechanism, and positioning of the consumable such that ophthalmic liquid is dispensed through the fluid outlet along a protected, standoff delivery path. Body 1 is intended for use with a properly configured consumable as defined herein. FIGS. 11 and 15 illustrate, in one embodiment, the consumable received within body 1 in the fixed operational position. Embodiments of the consumable are described in a companion application by the same inventor. The body-consumable interface thereby functions as a physical compatibility control configured to reduce the likelihood that a non-corresponding consumable is received, seated, aligned, retained, or actuated within internal cavity 27 of body 1, thereby supporting intended dose delivery, contamination control, and safety of the user.

As shown in FIG. 16, in one embodiment, the consumable includes exterior geometry features 3a and 3b on pod 3, exterior geometry feature 5a on flow-control hub 5, and exterior geometry feature 17a on protective cap 17. These exterior geometry features comprise alignment, keying, transition, and retention surfaces configured to complement predetermined interior geometry of body 1 and permit receipt only of a correspondingly configured consumable within body cavity 27.

In one embodiment, narrowed wall region 24 is configured to receive pod 3 in a predetermined orientation defined by exterior geometry feature 3a, and exterior geometry feature 3b provides a transition surface facilitating insertion of pod 3 past narrowed wall region 24. Exterior geometry feature 5a of flow-control hub 5 is configured to engage retention shelf 19 and corresponding interior geometry 26 so as to position the consumable at a predetermined depth within body cavity 27, prevent passage of pod 3 and an upper portion of flow-control hub 5 through exit port 18, inhibit rotation of the consumable within body cavity 27, and provide a stop for lateral alignment. A lower portion of flow-control hub 5 extends through exit port 18 and is configured to receive protective cap 17.

In one embodiment, body 1 and consumable 200 include corresponding visual compatibility indicia configured to provide visual confirmation that consumable 200 corresponds to body 1 before insertion, seating, or actuation. The visual compatibility indicia may be molded, embossed, printed, colored, textured, or otherwise formed on body 1 and on one or more of pod 3, flow-control hub 5, and protective cap 17. In one embodiment, the visual compatibility indicia is positioned to guide orientation of consumable 200 relative to body 1 and works together with the complementary geometry of body 1 and consumable 200 to support proper seating, alignment, actuation, dose delivery, contamination control, and safety of the user.

In other embodiments, body 1 may include a receiving structure configured to receive, support, hold, retain, guide, or align the replaceable consumable relative to the eye. The receiving structure may include an internal cavity, partial cavity, channel, cradle, slot, collar, clip, frame, external mount, or other support structure configured to position the replaceable consumable for actuation and dispensing.

In other embodiments, the actuation mechanism may be configured to actuate any suitable portion of the replaceable consumable to dispense ophthalmic liquid. For example, the actuation mechanism may deform, displace, compress, translate, pivot, depress, or otherwise actuate a pod, reservoir, hub, valve, membrane, plunger, piston, flexible wall, or other portion of the replaceable consumable.

In other embodiments, ophthalmic liquid may be dispensed through a fluid outlet associated with the reusable body, the replaceable consumable, or both. The fluid outlet may be formed on, carried by, coupled to, or placed in fluid communication with the reusable body, the replaceable consumable, a flow-control hub, a nozzle, a cannula, a conduit, or another fluid-directing structure.

In other embodiments, the replaceable consumable may include a protective structure configured to protect the fluid outlet, maintain cleanliness, reduce contamination risk, assist insertion or removal, or cooperate with the reusable body. The protective structure may include a cap, cover, seal, membrane, magnetized structure, valve, closure, collar, sleeve, sheath, or removable protective member.

In other embodiments, corresponding compatibility features between body 1 and the replaceable consumable may comprise mechanical, visual, tactile, magnetic, electronic, optical, or other compatibility-control features, or combinations thereof. Such compatibility features may be configured to guide insertion, confirm correspondence, control seating, inhibit rotation, prevent improper actuation, or otherwise support intended use of the replaceable consumable with body 1.

In other embodiments, the replaceable consumable may be retained relative to body 1 by any suitable retention structure. The retention structure may include a shelf, stop, shoulder, latch, detent, rib, groove, taper, friction fit, snap fit, bayonet engagement, magnetic stop, collar, cap, closure, or other structure configured to limit movement of the replaceable consumable relative to body 1 before, during, or after actuation.

In other embodiments, body 1 may include a stabilization structure configured to position the fluid outlet relative to the eye while maintaining spacing between the fluid outlet and ocular tissue. The stabilization structure may include one or more feet, pads, surfaces, legs, arms, bridges, frames, orbital supports, brow supports, cheek supports, nose supports, head-mounted supports, shoulder-supported supports, or other positioning structures.

In other embodiments, the replaceable consumable may comprise a pod, reservoir, cartridge, container, ampoule, vial, blister, or other liquid-holding structure configured to contain ophthalmic liquid and cooperate with body 1 for dispensing.

In other embodiments, body 1 may be configured for use with a single-use consumable, a multi-use consumable, a disposable consumable, a refillable consumable, or a replaceable consumable having a predetermined use period. The consumable may be removed, replaced, discarded, refilled, or serviced after depletion of the ophthalmic liquid or after a predetermined number of administrations.

In other embodiments, body 1 may be configured to receive the replaceable consumable through an entry port, side opening, rear opening, top opening, bottom opening, sliding channel, hinged opening, removable cover, cartridge bay, or other access structure configured to permit insertion or removal of the replaceable consumable.

In other embodiments, the replaceable consumable may be inserted, seated, retained, actuated, and removed by linear movement, sliding movement, rotational movement, pivoting movement, snap-in movement, bayonet movement, threaded movement, or combinations thereof relative to body 1.

In other embodiments, the replaceable consumable may be positioned relative to body 1 by one or more alignment structures configured to establish axial, lateral, rotational, depth, or outlet alignment of the replaceable consumable relative to the actuation mechanism, the fluid outlet, or the user's eye.

In other embodiments, the reusable body, the replaceable consumable, or both may include one or more safety-control structures configured to reduce unintended contact with ocular tissue, reduce contamination risk, inhibit use of a non-corresponding consumable, limit actuator travel, limit insertion depth, control outlet positioning, or support intended dispensing performance.

What is claimed is:

1. An ophthalmic delivery system comprising:
a reusable body defining an internal cavity configured to receive a replaceable consumable containing an ophthalmic liquid, the reusable body including a first foot and a second foot configured to stabilize the reusable body against a user's face adjacent an eye;
the replaceable consumable being removably received within the internal cavity and including a fluid outlet; and
an actuation mechanism carried by the reusable body and configured to transmit force to the replaceable consumable to cause ophthalmic liquid to be dispensed through the fluid outlet;
wherein, when the replaceable consumable is received within the internal cavity, the reusable body positions the fluid outlet to dispense ophthalmic liquid toward the eye along a protected, standoff delivery path.

2. The ophthalmic delivery system of claim 1, wherein the reusable body comprises a stabilization structure configured to position the fluid outlet relative to the eye while maintaining spacing between the fluid outlet and ocular tissue.

3. The ophthalmic delivery system of claim 1, wherein the reusable body is configured to be positioned relative to the eye by a support structure selected from the group consisting of a facial support, a head-mounted support, a shoulder-mounted support, and a frame support.

4. The ophthalmic delivery system of claim 1, wherein the reusable body includes one or more facial engagement surfaces configured to stabilize the reusable body against the user's face adjacent the eye.

5. The ophthalmic delivery system of claim 1, wherein the internal cavity includes predetermined interior geometry configured to receive the replaceable consumable in a fixed operational position.

6. The ophthalmic delivery system of claim 5, wherein the predetermined interior geometry is configured to cooperate with exterior geometry of the replaceable consumable to align the replaceable consumable with the actuation mechanism.

7. The ophthalmic delivery system of claim 5, wherein the predetermined interior geometry is configured to inhibit rotation of the replaceable consumable within the internal cavity.

8. The ophthalmic delivery system of claim 1, wherein the reusable body and the replaceable consumable include corresponding compatibility features configured to permit the replaceable consumable to be seated and actuated in a predetermined operational position.

9. The ophthalmic delivery system of claim 8, wherein the corresponding compatibility features comprise mechanical, visual, tactile, magnetic, electronic, optical, or combinations thereof.

10. The ophthalmic delivery system of claim 1, wherein the reusable body includes a receiving structure configured to receive, support, hold, retain, guide, or align the replaceable consumable.

11. The ophthalmic delivery system of claim 1, wherein the fluid outlet is carried by the reusable body, the replaceable consumable, or both.

12. The ophthalmic delivery system of claim 1, wherein the replaceable consumable includes a protective structure selected from the group consisting of a cap, cover, seal, membrane, valve, closure, collar, sleeve, sheath, and removable protective member.

13. The ophthalmic delivery system of claim 1, wherein the reusable body includes a locking structure configured to inhibit actuation of the actuation mechanism when not in use.

14. The ophthalmic delivery system of claim 1, wherein the reusable body includes an entry port through which the replaceable consumable is insertable into and removable from the internal cavity.

15. The ophthalmic delivery system of claim 14, wherein the reusable body includes an exit port through which at least a portion of the replaceable consumable extends when the replaceable consumable is received within the internal cavity.

16. The ophthalmic delivery system of claim 15, wherein the exit port has a predetermined geometry configured to admit a correspondingly configured protective cap of the replaceable consumable.

17. The ophthalmic delivery system of claim 1, wherein the reusable body includes a retention structure configured to engage the replaceable consumable and limit insertion depth of the replaceable consumable within the internal cavity.

18. The ophthalmic delivery system of claim 17, wherein the retention structure comprises a shelf, stop, shoulder, latch, detent, rib, groove, taper, friction fit, snap fit, bayonet engagement, magnetic stop, collar, closure, or combinations thereof.

19. The ophthalmic delivery system of claim 17, wherein the retention structure includes interior geometry configured to cooperate with exterior geometry of the replaceable consumable to inhibit rotation of the replaceable consumable within the internal cavity.

20. The ophthalmic delivery system of claim 1, wherein the reusable body includes a narrowed wall region configured to receive the replaceable consumable in a predetermined orientation.

21. The ophthalmic delivery system of claim 20, wherein the narrowed wall region is configured to cooperate with a flat exterior surface of the replaceable consumable.

22. The ophthalmic delivery system of claim 20, wherein the replaceable consumable includes a transition surface configured to facilitate insertion of the replaceable consumable past the narrowed wall region.

23. The ophthalmic delivery system of claim 1, wherein the reusable body and the replaceable consumable include corresponding visual compatibility indicia configured to provide visual confirmation that the replaceable consumable is configured for use with the reusable body before insertion, seating, or actuation.

24. The ophthalmic delivery system of claim 1, wherein the actuation mechanism is positioned at an upper portion of the reusable body and is movable between an open position permitting insertion or removal of the replaceable consumable and a closed position for dispensing ophthalmic liquid.

25. The ophthalmic delivery system of claim 1, wherein the actuation mechanism is positioned on a lateral side of the reusable body and is configured to deform under manual pressure and return toward a resting position upon release.

26. The ophthalmic delivery system of claim 1, wherein the actuation mechanism comprises a living hinge configured to deflect inward under manual pressure and return toward a resting position upon release.

27. The ophthalmic delivery system of claim 26, wherein the living hinge includes a protrusion configured to engage and deform the replaceable consumable during actuation.

28. The ophthalmic delivery system of claim 27, wherein the reusable body includes a limiting feature configured to limit travel of the living hinge during actuation.

29. The ophthalmic delivery system of claim 1, wherein the actuation mechanism is positioned on the reusable body at a location selected to transmit force to the replaceable consumable when the replaceable consumable is received within the internal cavity.

30. The ophthalmic delivery system of claim 1, wherein the replaceable consumable comprises a pod configured to contain the ophthalmic liquid and a flow-control hub coupled to the pod.

31. The ophthalmic delivery system of claim 30, wherein the flow-control hub includes the fluid outlet.

32. The ophthalmic delivery system of claim 30, wherein the pod is formed from a resilient deformable material.

33. The ophthalmic delivery system of claim 1, wherein the replaceable consumable is insertable into the internal cavity by a user prior to use and removable from the internal cavity after use.

34. The ophthalmic delivery system of claim 1, wherein the reusable body has an arched configuration defining a first leg terminating in the first foot and a second leg terminating in the second foot.

35. The ophthalmic delivery system of claim 34, wherein the first foot is configured to contact the user's face above the eye and the second foot is configured to contact the user's face below the eye.

36. The ophthalmic delivery system of claim 34, wherein the first leg is shorter than the second leg.

37. The ophthalmic delivery system of claim 1, wherein the reusable body is configured to position the fluid outlet in a recessed relationship relative to the first foot and the second foot such that the fluid outlet does not extend beyond a plane tangential to both the first foot and the second foot when the replaceable consumable is received within the internal cavity.

38. The ophthalmic delivery system of claim 1, wherein the reusable body is configured to maintain the fluid outlet spaced away from the ocular surface, eyelashes, and surrounding periocular tissue during use.

39. A method of administering an ophthalmic liquid, comprising:
inserting a replaceable consumable containing ophthalmic liquid into an internal cavity of a reusable body;
positioning a first foot and a second foot of the reusable body against a user's face adjacent an eye;
actuating an actuation mechanism carried by the reusable body to transmit force to the replaceable consumable and dispense ophthalmic liquid through a fluid outlet of the replaceable consumable toward the eye along a protected, standoff delivery path; and
removing the replaceable consumable from the reusable body after use.

40. The method of claim 39, wherein inserting the replaceable consumable comprises advancing the replaceable consumable through an entry port of the reusable body.

41. The method of claim 39, wherein inserting the replaceable consumable comprises seating the replaceable consumable against a retention shelf of the reusable body.

42. The method of claim 39, wherein inserting the replaceable consumable comprises aligning exterior geometry of the replaceable consumable with predetermined interior geometry of the internal cavity.

43. The method of claim 39, wherein actuating the actuation mechanism comprises deforming a pod of the replaceable consumable.

44. The method of claim 39, wherein removing the replaceable consumable comprises removing an entry port cap and advancing the replaceable consumable until a portion of the replaceable consumable protrudes from the reusable body.

45. A reusable body for an ophthalmic delivery system, comprising:
a body structure defining an internal cavity configured to receive a replaceable consumable containing an ophthalmic liquid;
a first foot and a second foot configured to stabilize the body structure against a user's face adjacent an eye; and
an actuation mechanism carried by the body structure and configured to transmit force to the replaceable consumable when the replaceable consumable is received within the internal cavity;
wherein the body structure is configured to position a fluid outlet of the replaceable consumable to dispense ophthalmic liquid toward the eye along a protected, standoff delivery path.

46. The reusable body of claim 45, wherein the internal cavity includes predetermined interior geometry configured to receive the replaceable consumable in a fixed operational position.

47. The reusable body of claim 46, wherein the predetermined interior geometry is configured to align the replaceable consumable with the actuation mechanism.

48. The reusable body of claim 46, wherein the predetermined interior geometry is configured to inhibit rotation of the replaceable consumable within the internal cavity.

49. The reusable body of claim 45, further comprising an entry port through which the replaceable consumable is insertable into and removable from the internal cavity.

50. The reusable body of claim 49, further comprising an exit port through which at least a portion of the replaceable consumable extends when received within the internal cavity.

51. The reusable body of claim 50, wherein the exit port has a predetermined geometry configured to admit a correspondingly configured protective cap of the replaceable consumable.

52. The reusable body of claim 45, further comprising a retention shelf configured to engage the replaceable consumable and limit insertion depth of the replaceable consumable within the internal cavity.

53. The reusable body of claim 52, wherein the retention shelf includes interior geometry configured to cooperate with exterior geometry of the replaceable consumable to inhibit rotation of the replaceable consumable within the internal cavity.

54. The reusable body of claim 45, wherein the internal cavity includes a narrowed wall region configured to receive the replaceable consumable in a predetermined orientation.

55. The reusable body of claim 45, wherein the actuation mechanism is selected from the group consisting of a top-mounted actuator, a side-mounted actuator, and a living hinge actuator.

56. The reusable body of claim 45, wherein the body structure has an arched configuration defining a first leg terminating in the first foot and a second leg terminating in the second foot.

57. The reusable body of claim 56, wherein the first leg is shorter than the second leg.

58. The reusable body of claim 45, wherein the body structure is configured to maintain the fluid outlet of the replaceable consumable spaced from the ocular surface during use.

59. The reusable body of claim 45, wherein the body structure includes visual compatibility indicia configured to correspond to visual compatibility indicia on the replaceable consumable.

60. The reusable body of claim 45, wherein the body structure includes a locking structure configured to inhibit actuation of the actuation mechanism when not in use.
